(19) United States
(12) Patent Application Publication
(10) Pub. No.: US 2023/0130762 A1
(43) Pub. Date: Apr. 27, 2023

(54) PLASMA GENERATING APPARATUS AND LARGE-VOLUME PLASMA TREATMENT SYSTEM

(71) Applicant: FERMION INSTRUMENTS (SHANGHAI) CO., LTD., Shanghai (CN)

(72) Inventors: Binping XIE, Shanghai (CN); Yanbian FANG, Shanghai (CN); Liang WANG, Shanghai (CN); Jiayue GUAN, Shanghai (CN); Yanhui WANG, Shanghai (CN)

(73) Assignee: FERMION INSTRUMENTS (SHANGHAI) CO., LTD., Shanghai (CN)

(21) Appl. No.: 17/913,444

(22) PCT Filed: Mar. 24, 2021
(86) PCT No.: PCT/CN2021/082714

§ 371 Date: Sep. 22, 2022

(30) Foreign Application Priority Data
Mar. 25, 2020 (CN) 202010218844.9

(51) Int. Cl. A61L 2/14; A61L 2/24; A61L 2/26

(52) U.S. Cl. CPC A61L 2/14; A61L 2/24; A61L 2/26; A61L 2202/11; A61L 2202/122; A61L 2202/14

(57) ABSTRACT

A plasma generating apparatus includes a feed-through and a cylindrical electrode. The feed-through device is configured to feed a medium frequency (MF) power. The cylindrical electrode includes a plurality of through holes in a wall of the cylindrical electrode and is connected to the feed-through device and configured to receive the MF power from the feed-through device and ionize a gas to generate plasma. The cylindrical electrode includes a main body; and an end cover fixed to one end of the main body. The feed-through device includes a feed-through conductor electrically connected to the cylindrical electrode and configured to feed the MF power to the cylindrical electrode, and a feed-through insulating layer covering at least a part of the feed-through conductor.

### CROSS-REFERENCE TO RELATED APPLICATION

The present application claims priority to Chinese Patent Application No. 202010218844.9, entitled “LARGE-VOLUME VACUUM PLASMA STERILIZATION AND DISINFECTION DEVICE”, filed on Mar. 25, 2020, which is incorporated herein by reference in its entirety.

### TECHNICAL FIELD

The present disclosure relates to the field of plasma and, in particular, to a plasma generating apparatus and a plasma treatment system.

### BACKGROUND

Plasma has been widely applied in many fields. For example, low-temperature plasma has a broad application prospect in sterilization and disinfection.

At present, conventional sterilization and disinfection technologies applied in biological and medical industries mainly include a high-temperature method, a chemical soaking or wiping method (e.g., formalin, chlorine-containing disinfectants, ethanol, iodine solutions, and hydrogen peroxide), a low-temperature fumigation method (e.g., ethylene oxide (EO) and low-temperature formaldehyde vapor), and a radiation method (e.g., ultraviolet rays and gamma rays).

However, conventional sterilization and disinfection technologies all have defects or restrictions. For the high-temperature sterilization and disinfection method, many materials in modern medical devices are thermo-sensitive without resistance to high temperatures. The chemical soaking or wiping method is not suitable for hygroscopic materials and has weak penetration, leading to incomplete disinfection. For the EO fumigation method, EO is a flammable, combustible, and cancerogenic substance. The EO fumigation method has a long sterilization time (e.g., more than 6 h), and residues must be separated and treated for 14 d after sterilization. For the low-temperature formaldehyde vapor method, formaldehyde is cancerogenic, likely to generate residues and pollution, and also the low-temperature formaldehyde vapor method has a long sterilization time (e.g., 4-6 h). The radiation sterilization and disinfection method has a small coverage area on the article due to the linear propagation of radiation and cannot completely sterilize complicated articles (e.g., concave structure).

A lot of studies have revealed that low-temperature plasma has sterilization and disinfection functions, such as Hideharu Shintani, Akikazu Sakudo, Peter Burke and Gerald Mcdonnell, Experimental and Therapeutic Medicine 1,731-738 (2010), Hideharu Shintani and Akikazu Sakudo, Gas Plasma Sterilization in Microbiology (Caister Academic Press, Norfolk, 2016), p.1-40 and Rossi F, Kylian Oand Hasiwa M, Plasma Process Polym 3,431-442 (2006). The plasma can be used for etching. Neutral activated particles in the plasma can chemically react with proteins and nucleic acids of the microorganism, interfering with the survival function of the microorganism or even destroying the microorganism, thereby achieving the sterilization and disinfection effect.

However, based on conventional plasma technologies, it is hard to manufacture large-volume sterilization and disinfection devices. The plasma is commonly obtained by high-voltage discharge, but high-temperature spark or arc discharge are easily formed by directly ionizing a gas between two metal electrodes, which is disadvantageous for applications in the biological and medical fields. Moreover, the low-temperature plasma is typically obtained by dielectric barrier discharge (DBD), surface discharge, plasma jet, etc. For the DBD and the surface discharge, an insulating layer is provided between electrodes to prevent the breakdown of the electrodes, but a small working distance between the two electrodes poses great difficulty in manufacturing large-volume sterilization and disinfection devices. The plasma jet is also hardly applied to large-volume sterilization and disinfection devices due to the limited distance of the jetted plasma.

A hydrogen peroxide plasma sterilizer is another sterilization device using plasma. The sterilizer mainly uses a sterilization effect of hydrogen peroxide vapor. Specifically, highly active hydroxyls in the hydrogen peroxide are acted on cytomembranes, destroying proteins of bacteria with strong oxidation and killing the bacteria (similar to the sterilization principle of the potassium permanganate solution). However, the plasma formed by ionization is used to remove residual hydrogen peroxide, rather than using the etching effect of the plasma for sterilization and disinfection. Therefore, with the assistance of the plasma for sterilization and disinfection, the hydrogen peroxide plasma sterilizer has a limited killing effect on bacterial spores.

### SUMMARY

An embodiment of the present disclosure provides a plasma generating apparatus including a feed-through device and a cylindrical electrode. The feed-through device is configured to feed a medium frequency (MF) power. The cylindrical electrode includes a plurality of through holes in the wall of the cylindrical electrode and is connected to the feed-through device and configured to receive the MF power from the feed-through device and ionize a gas to generate plasma.

An embodiment of the present disclosure provides a plasma treatment system that includes at least one plasma generating apparatus that includes a feed-through device, a cylindrical electrode, and a plasma treatment chamber. The feed-through device is configured to feed an MF power. The cylindrical electrode includes a plurality of through holes in a wall of the cylindrical electrode and is connected to the feed-through device and configured to receive the MF power from the feed-through device and ionize a gas to generate plasma. The plasma treatment chamber communicates with at least one plasma generating apparatus, such that the plasma is diffused to the plasma treatment chamber.

### BRIEF DESCRIPTION OF THE DRAWINGS

To describe the technical solutions of the embodiments of the present disclosure more clearly, accompanying drawings of the embodiments will be briefly introduced below. It is to be understood that these drawings are merely for example, rather than any limitation to the embodiments of the present disclosure.

FIG. 1 is a sectional view of a plasma generating apparatus according to an embodiment of the present disclosure.

FIG. 2A and FIG. 2B each are perspective views of a cylindrical electrode according to an embodiment of the present disclosure.

FIG. 3 is a structural view of a plasma treatment system including a plasma generating apparatus according to an embodiment of the present disclosure.

FIG. 4 is a structural view of a plasma treatment system including a plurality of plasma generating apparatuses according to an embodiment of the present disclosure.

### REFERENCE NUMERALS

100 and 100a-f plasma generating apparatus

101 feed-through conductor

102 cylindrical electrode

1021 through hole

1022 water cooling channel

1023 main body

1024 end cover

1025 connecting hole

1026a water inlet

1026b water outlet

103 feed-through insulating layer

104 bolt

105 housing

106 plasma generating chamber

107 water cooling channel

108a water outlet

108b water inlet

109 gas inlet

110 base

111a water inlet tube

111b water outlet tube

112a-112b insulating layer

113 power supply

114 controller

300 and 400 plasma treatment system

310 and 410 gas supply device

311 and 411 gas source

312 and 412 filter

313 and 413 needle valve

314 and 414 gas inlet valve

320 and 420 plasma treatment chamber

321 and 421 chamber door

322 and 422 vacuum gauge

330 and 430 vacuum device

331 and 431 vacuum pump

332 and 432 vacuum valve

340 and 440 vacuum release device

341 and 441 filter

342 and 442 gas release valve

### DETAILED DESCRIPTION OF THE EMBODIMENTS

Some embodiments of the present disclosure will be described below with reference to the drawings. The described embodiments are merely exemplary embodiments rather than all of the embodiments of the present disclosure.

It should be noted that in the description of the present disclosure, the terms, such as “center”, “upper”, “lower”, “left”, “right”, “vertical”, “horizontal”, “inner” and “outer,” indicate the orientation or position relationships based on the drawings. These terms are merely intended to facilitate the description of the present disclosure and simplify the description, rather than to indicate or imply that the mentioned apparatus or element must have a specific orientation and must be constructed and operated in a specific orientation. Therefore, these terms should not be construed as a limitation to the present disclosure. Moreover, terms, such as “first” and “second,” are merely intended for the purpose of description and should not be construed as indicating or implying relative importance. In the description of the present disclosure, it should be noted that, unless otherwise clearly specified, meanings of the terms “install”, “connected with”, “connected to”, and “coupled to” should be understood in a broad sense. For example, the connection may be a fixed connection or a removable connection; a mechanical connection or an electrical connection; a direct connection or an indirect connection by using an intermediate medium; or intercommunication between two components. Those of ordinary skill in the art may understand specific meanings of the foregoing terms in the present disclosure based on a specific situation.

It should be understood by those skilled in the art that the embodiments of the present disclosure can be widely applied in various fields. In the description of the present disclosure, examples of biological and medical fields are merely for simple and clear description, rather than as a limitation to the embodiments of the present disclosure. Conversely, the embodiments of the present disclosure can be applied in other fields, such as material processing, semiconductors, cold-chain logistics, and fresh food processing. It should be understood by those skilled in the art that the embodiments of the present disclosure can be applied to various scenarios, including but not limited to, sterilization, disinfection, cleaning, etching, surface treatment, etc.

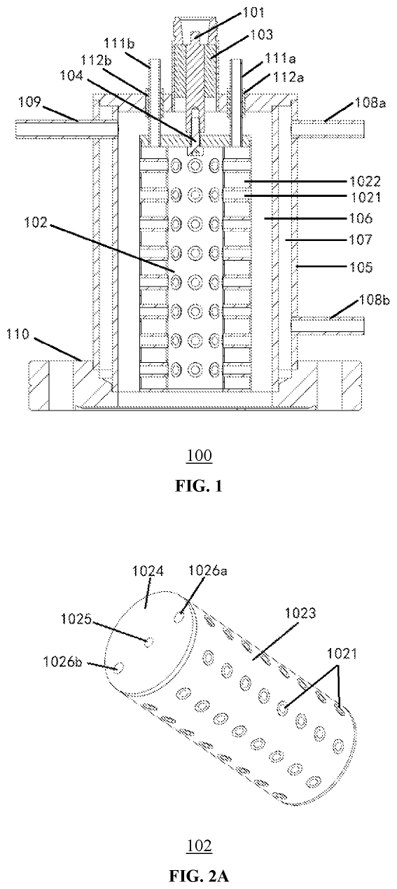

FIG. 1 is a sectional view of plasma generating apparatus 100 according to an embodiment of the present disclosure. As shown in FIG. 1, the plasma generating apparatus 100 may include a feed-through device and cylindrical electrode 102 connected to the feed-through device. The feed-through device may feed power to the cylindrical electrode 102, such as a radio frequency (RF) power or an MF power. In some embodiments, the RF power or the MF power may be characterized by other physical variables, such as a voltage, a current, or a frequency.

In some embodiments, the feed-through device may include a feed-through conductor 101. The feed-through conductor 101 may be electrically connected to the cylindrical electrode 102 and configured to feed power. As shown in FIG. 1, the feed-through conductor 101 may be fixedly connected to the cylindrical electrode 102 through bolt 104. The feed-through conductor 101 may come into contact with the cylindrical electrode 102 to form a conductive path. Moreover, the bolt 104 may include a conductive material to form the conductive path between the feed-through conductor 101 and the cylindrical electrode 102. It should be understood that the feed-through conductor 101 and the cylindrical electrode 102 may be connected in other manners, such as bonding and welding.

In some embodiments, the feed-through device may include feed-through insulating layer 103. The feed-through insulating layer 103 covers at least a part of the feed-through conductor 101 and may insulate the feed-through conductor 101 from the outside (e.g., housing 105). The feed-through insulating layer 103 may include various insulating materials, such as a ceramic material.

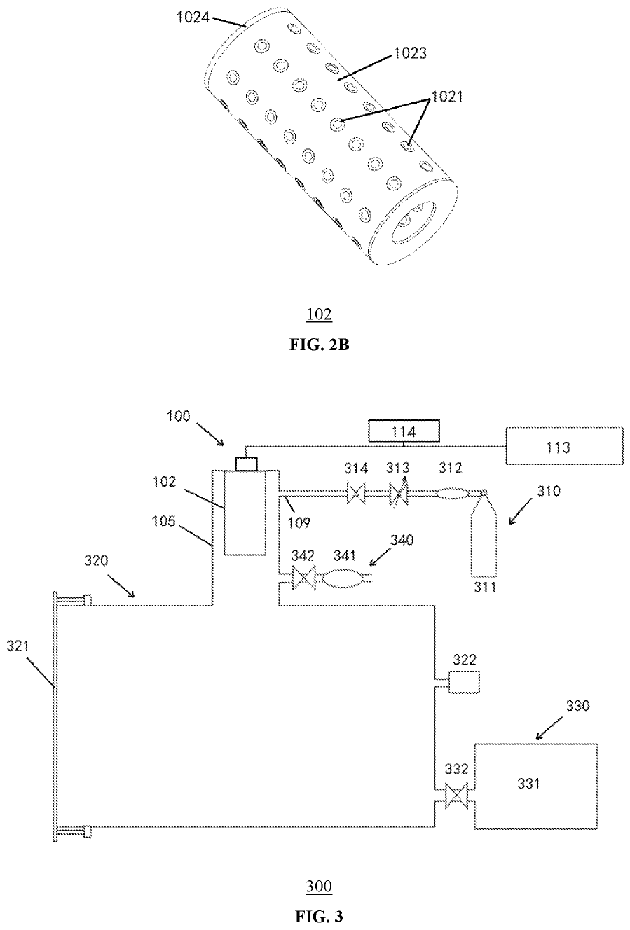

The cylindrical electrode 102 may include a plurality of through holes 1021 in the wall of the cylindrical electrode. FIG. 2A and FIG. 2B are perspective views of a cylindrical electrode 102 according to an embodiment of the present disclosure. As shown in FIG. 2A and FIG. 2B, the cylindrical electrode 102 may include main body 1023 and end cover 1024. In some embodiments, the main body 1023 may be formed integrally or by curling a flat plate. The main body 1023 may have any suitable cross-sectional shape, such as a circle, a rectangle, a polygon, or an ellipse. The main body 1023 includes the plurality of through holes 1021 in the wall. The through holes 1021 each may have any suitable shape, such as a circle, a rectangle, a polygon, or an ellipse.

The main body 1023 may include a conductive material, such as stainless steel, tantalum, aluminum, titanium, molybdenum, niobium, tungsten, and graphite. Aluminum materials may not be suitable for a high-power plasma actuator due to the low melting point and no high-temperature resistance. Because of the high melting point, high-temperature resistance, and small electron work function, titanium, molybdenum, and niobium materials are more suitable for the high-power plasma actuator with high ionization efficiency. It should be understood that the main body 1023 may include other nonmagnetic materials with a small work function and high-temperature resistance.

The end cover 1024 may be fixed on the main body 1023. For example, the end cover 1024 may be formed integrally with the main body 1023, or the end cover 1024 may be welded on the main body 1023. The end cover 1024 may include a material that is the same as or different from that of the main body 1023.

In some embodiments, the end cover 1024 may be fixedly connected to the feed-through conductor 101. For example, the feed-through conductor 101 may be fixedly connected to the end cover 1024 at a central position of the end cover 1024. As shown in FIG. 2A, the end cover 1024 may include connecting hole 1025 (e.g., connecting hole 1025 at the center of the end cover 1024). Moreover, the connecting hole 1025 may include a screw thread. The bolt 104 may connect the feed-through conductor 101 and the cylindrical electrode 102 fixedly through the connecting hole 1025, as shown in FIG. 1.

The cylindrical electrode 102 may ionize a gas to generate plasma. The gas may include air, oxygen, nitrogen, an inert gas, liquid vapor, etc. During operation, the cylindrical electrode 102 may form a hollow cathode discharge in which a pendulum motion of electrons occurs. Under the actuation of a power supply, electrons escaping from one side of the cylindrical electrode 102 are accelerated by an electric field on the same side. If enough energy is obtained to enter a cathode fall region on the other side, the electrons are repelled by an electric field at the other side and then returned. In this way, the electrons pendulate back and forth between two sides of a cylinder, which increases the possibility that neutral particles are excited and ionized and can greatly improve the efficiency and capability of the plasma actuator.

The existing plasma generating apparatus typically uses an RF actuator. However, due to a low automatic bias of the RF actuator, the power of the plasma generating apparatus is hardly improved. Moreover, the matching problem will become tricky in the RF actuator and lead to instability. To address the matching problem, the plasma generating apparatus using the RF actuator is provided with a network matcher, such as a vacuum capacitance matcher.

In some embodiments, the plasma generating apparatus 100 may use an MF actuator. The feed-through device (e.g., the feed-through conductor 101) may feed an MF power to the cylindrical electrode 102. The MF power may have a frequency in the range of 20 kHz-100 kHz. The MF actuator features a high automatic bias and a high power. For example, the automatic bias may be in the range of 400 V-100 V, and the power may be at least 1,000 W, such that actuated ions have high energy. In some embodiments, the plasma generating apparatus 100 may be applied to high-power and large-volume vacuum treatment devices.

In some embodiments, the plasma generating apparatus 100 may implement load matching without the network matcher. Therefore, the plasma generating apparatus 100 has higher stability and lower cost.

In some embodiments, as shown in FIG. 1, the plasma generating apparatus 100 may include housing 105. The housing 105 may define plasma generating chamber 106. The cylindrical electrode 102 is provided in the plasma generating chamber 106. The feed-through device may be provided on the housing 105, for example, in an opening of the housing 105, as shown in FIG. 1. The feed-through conductor 101 may extend to the plasma generating chamber 106 through the housing 105 and is connected to the cylindrical electrode 102. The feed-through insulating layer 103 may be provided between the feed-through conductor 101 and the housing 105 and insulate the feed-through conductor 101 from the housing 105.

In some embodiments, as shown in FIG. 1, the housing 105 may include gas inlet 109 communicating with the plasma generating chamber 106 and configured to supply gas to the plasma generating chamber 106. The gas may include one or more selected from the group consisting of air, oxygen, nitrogen, an inert gas, and liquid vapor.

In some embodiments, as shown in FIG. 1, the housing 105 may include water cooling channel 107 as well as water outlet 108a and water inlet 108b communicating with the water cooling channel 107. It should be understood that the water outlet 108a and the water inlet 108b may be interchangeable to form the water inlet 108a and the water outlet 108b. The water cooling channel 107 may be provided in a sidewall of the housing 105. For example, the sidewall of the housing 105 includes a sandwich structure, and the water cooling channel 107 is provided in the sandwich structure, as shown in FIG. 1. Alternatively, the sidewall of the housing 105 may be provided with a water cooling tube to form the water cooling channel 107. The water inlet 108b receives cooling water from the outside. The cooling water flows through the water cooling channel 107, for example, under the driving of pressure, and flows out of the water outlet 108a, thereby exchanging heat with the housing 105 to cool the plasma generating apparatus 100.

In some embodiments, the cylindrical electrode 102 may include water cooling channel 1022, as shown in FIG. 1. For example, the cylindrical electrode 102 may include an inner layer, an outer layer, and the water cooling channel 1022 between the inner layer and the outer layer. A sidewall of each of the through holes 1021 may be sealingly connected to the inner layer and the outer layer, for example by bonding, welding, or integral forming. As shown in FIG. 2A, the cylindrical electrode 102 further includes water inlet 1026a and water outlet 1026b in the end cover 1024. Both the water inlet 1026a and the water outlet 1026b communicate with the water cooling channel 1022. As shown in FIG. 1, the plasma generating apparatus 100 may further include water inlet tube 111a and water outlet tube 111b. The water inlet tube 111a is connected to the water inlet 1026a of the cylindrical electrode 102 and configured to supply the cooling water to the cylindrical electrode 102. The water outlet tube 111b is connected to the water outlet 1026a of the cylindrical electrode 102 and configured to discharge the cooling water from the cylindrical electrode 102. Both the water inlet tube 111a and the water outlet tube 111b may be connected to an external water supply device through the housing 105. In some embodiments, both the water inlet tube 111a and the water outlet tube 111b may include a metal tube respectively connected to the water inlet 1026a and the water outlet 1026b and are insulated from the housing 105 through insulating layers 112a and 112b. At least a part of each of the water inlet tube 111a and the water outlet tube 111b may include an insulating tube (such as a plastic tube) for insulating the cylindrical electrode 102.

During operation, the cooling water enters the water cooling channel 1022 through the water inlet tube 111a and the water inlet 1026a to exchange heat with the cylindrical electrode 102 and then flows out of the water outlet 1026b and the water outlet tube 111b. In some embodiments, the cylindrical electrode 102 maintains the temperature (i.e., the temperature remains unchanged) to improve the stability of ionization.

It should be understood that although the above description uses cooling water, the present disclosure is not limited thereto and may also use other condensed liquids. It should be further understood that the cylindrical electrode 102 or the housing 105 may include other cooling devices, such as a fan.

In some embodiments, the plasma generating apparatus 100 may work at high power under the cooling effect of the water cooling channel 107 and/or the water cooling channel 1022. For example, the plasma generating apparatus 100 may work for a long time at no less than 100 W or no less than 1,000 W or even at a few kilowatts.

In some embodiments, as shown in FIG. 1, the plasma generating apparatus 100 may include base 110 fixedly connected to the housing 105. The base 110 may support and fix the plasma generating apparatus 100. For example, the base 110 can fix the plasma generating apparatus 100 to a device having a larger volume (such as a vacuum chamber).

In some embodiments, the base 110 may include an opening that communicates with the plasma generating chamber 106 and a connecting flange. For example, the base 110 may include a CF flange. The CF flange can connect the plasma generating apparatus 100 to a high vacuum device and an ultra-high vacuum device to realize sealing at a low leakage rate. The base 110 may include other types of flanges, such as a KF flange, an ISO-K flange, or an ISO-F flange.

In some embodiments, the plasma generating apparatus 100 may be a vacuum device. The plasma generating chamber 106 may be located in a vacuum atmosphere. In the case of the MF actuator, the plasma generating apparatus 100 may start (turn on) the plasma actuator at very low pressure. For example, the pressure at which the plasma actuator of the plasma generating apparatus 100 is started (turned on) may be as low as about 7 Pa.

FIG. 3 is a structural view of plasma treatment system 300 according to an embodiment of the present disclosure. The plasma treatment system 300 may include a plasma generating apparatus (such as plasma generating apparatus 100 shown in FIG. 1). The plasma generating apparatus 100 may include cylindrical electrode 102 and housing 105. The plasma generating apparatus 100 may further include power supply 113 and controller 114. The power supply 113 may include an RF power or an MF power. The power supply 113 may be connected to a feed-through device and configured to feed the RF power or the MF power to the cylindrical electrode 102. The controller 114 may be connected to the power supply 113 and/or the feed-through device and configured to control the plasma generating apparatus 100. It should be understood that although the controller 114 and the power supply 113 are separated in FIG. 3, the controller 114 and the power supply 113 can be integrated into a single device. The controller 114 may control the on-off of the plasma generating apparatus 100, the frequency of the power supply 113, etc.

In some embodiments, as shown in FIG. 3, the plasma treatment system 300 may include gas supply device 310. The gas supply device 310 may include gas source 311, such as a gas storage tank and a gas transmission tube. The gas source 311 may be connected to gas inlet 109 of the plasma generating apparatus 100 to communicate with the plasma generating chamber 106. The gas source 311 may supply gas to the plasma generating chamber 106, such as one or more selected from the group consisting of air, oxygen, nitrogen, an inert gas, and liquid vapor. The gas supply device 310 can provide different gases for the plasma generating apparatus 100 according to different application scenarios (such as the type and the number of articles to be treated by the plasma).

In some embodiments, as shown in FIG. 3, the gas supply device 310 may further include filter 312 between the gas source 311 and the gas inlet 109. The filter 312 can filter the gas from the gas source 311 to filter possible dust particles and impurities and improve the purity of the gas entering the plasma generating chamber 106. In this way, the stability of the plasma generating apparatus 100 can be improved to prolong the service life. For example, in some embodiments, the filter 312 is in a filter class for filtering particle sizes of at least 3 nm. Therefore, the filter 312 can filter the dust particles and possible bacteria and viruses in the gas, such that the article to be treated by the plasma, like a medical appliance and a protective article, is not polluted.

In some embodiments, as shown in FIG. 3, the gas supply device 310 may further include needle valve 313 and gas inlet valve 314 between the gas source 311 and the gas inlet 109. The needle valve 313 and the gas inlet valve 314 may be configured to control the flow velocity of the gas from the gas source. The needle valve 313 and the gas inlet valve 314 can maintain the plasma generating chamber 106 at an appropriate pressure by adjusting the flow velocity of the gas to generate the plasma. Similarly, the needle valve 313 and the gas inlet valve 314 can further maintain plasma treatment chamber 320 at an appropriate pressure to perform plasma treatment (such as sterilization, disinfection, and surface treatment) on an article in the plasma treatment chamber 320. The pressure to be maintained can be determined according to different factors, such as the gas and the article.

In some embodiments, the plasma treatment system 300 may include the plasma treatment chamber 320, as shown in FIG. 3. The plasma treatment chamber 320 may include a space for accommodating the article to be treated by the plasma. The plasma generating apparatus 100 is fixedly connected to the plasma treatment chamber 320, for example, through a flange of base 110. The plasma generating chamber 106 of the plasma generating apparatus 100 communicates with the accommodating space of the plasma treatment chamber 320. During operation, the plasma is generated in the plasma generating chamber 106. Charged particles and neutral activated particles (such as neutral excited state atoms, molecules, and free radicals) in the plasma are separated. The neutral activated particles can be diffused to the plasma treatment chamber 320 to perform the plasma treatment on the article in the accommodating space.

In some embodiments, as shown in FIG. 3, the plasma treatment chamber 320 may include chamber door 321. The article to be treated by the plasma may enter or exit the plasma treatment chamber 320 through the chamber door 321. In some embodiments, the chamber door 321 may use a fast-door design. The fast-door design is an informal term for vacuum appliance-related technologies. The difference between internal and external pressures forms a sealing effect with a bolt rather than a rubber seal ring. Through the chamber door 321 using the fast-door design, the article to be treated by the plasma is put into and taken out of the chamber conveniently, while the desired sealing effect can be kept even with frequent plasma treatment.

In some embodiments, as shown in FIG. 3, the plasma treatment chamber 320 may include vacuum gauge 322 configured to detect a vacuum degree in the plasma treatment chamber 320.

In some embodiments, the plasma treatment system 300 may include vacuum device 330, as shown in FIG. 3. The vacuum device 330 may include vacuum pump 331 and vacuum valve 332. The vacuum pump 331 is connected to the plasma treatment chamber 320 to communicate with the accommodating space of the plasma treatment chamber 320. The vacuum pump 331 may control a vacuum degree in the accommodating space of the plasma treatment chamber 320. The vacuum valve 332 is provided between the vacuum pump 331 and the plasma treatment chamber 320 and configured to control the on-off of the vacuum pump. In some embodiments, the vacuum device 330 can keep the vacuum degree in the plasma treatment chamber 320 at an mbar magnitude. The vacuum device 330 can accurately control the pressure in the plasma treatment chamber 320 to better perform the plasma treatment (such as sterilization and disinfection) on the article.

In some embodiments, the plasma treatment system 300 may include vacuum release device 340, as shown in FIG. 3. The vacuum release device 340 may be connected to the plasma generating apparatus 100 and may also be connected to the plasma treatment chamber 320. The vacuum release device 340 may include gas release valve 342 configured to control the opening and closing of a gas release channel. In some embodiments, the vacuum release device 340 may include a filter 341 configured to filter a gas entering the plasma generating apparatus 100 or the plasma treatment chamber 320 from the vacuum release device 340. The filter 341 can filter possible dust particles and impurities in the gas, which can improve the stability of the plasma treatment system 300 to prolong its service life and prevent secondary pollution to the article in the plasma treatment chamber 320. For example, in some embodiments, the filter 341 is in a filter class corresponding to a particle size of at least 3 nm. Therefore, the filter 341 can filter the dust particles, bacteria, and viruses in the gas entering the plasma generating apparatus 100 or the plasma treatment chamber 320 to prevent the secondary pollution to the article to be treated by the plasma.

In some embodiments, the plasma treatment system 300 may include a system controller (not shown). It should be understood that the system controller may be integrated with the controller 114, or the controller 114 may serve as the system controller. The system controller may be connected to one or more components of the plasma treatment system 300 and configured to control the connected component. For example, the system controller may be connected to the plasma generating apparatus 100 and the gas supply device 310 and configured to control the start and maintenance of the plasma actuator. When the pressure in the plasma generating chamber 106 or the plasma treatment chamber 320 is lower than a preset value (for example, a single preset value or a lower limit of a pressure range), the system controller opens the gas inlet valve 314 or increases an opening of the needle valve 313. When the pressure in the plasma generating chamber 106 or the plasma treatment chamber 320 is higher than a preset value (for example, a single preset value or an upper limit of a pressure range), the system controller closes the gas inlet valve 314 or decreases an opening of the needle valve 313. In this way, the system controller can maintain the pressure in the plasma generating chamber 106 or the plasma treatment chamber 320 at the preset value or within the preset range.

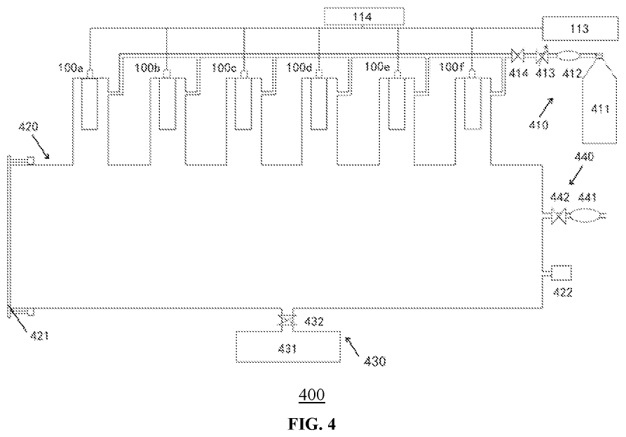

FIG. 4 is a structural view of a plasma treatment system 400 according to an embodiment of the present disclosure. The plasma treatment system 400 may include a plurality of plasma generating apparatuses, such as plasma generating apparatuses 100a-100f. The plasma generating apparatuses 100a-100f can be realized by the plasma generating apparatus 100 in FIG. 1. The plasma generating apparatuses 100a-100f each may include cylindrical electrode 102 and housing 105. The plasma generating apparatuses 100a-100f each may further include power supply 113 and controller 114.

In some embodiments, as shown in FIG. 4, the plasma generating apparatuses 100a-100f may share the power supply 113 and the controller 114. The power supply 113 may include an RF power or an MF power. The power supply 113 may be connected to a feed-through device of each of the plasma generating apparatuses 100a-100f and configured to feed the RF power or the MF power to the cylindrical electrode 102. The controller 114 may be connected to the power supply 113 and/or the feed-through device and configured to control each of the plasma generating apparatuses 100a-100f. It should be understood that although the controller 114 and the power supply 113 are separated in FIG. 4, the controller 114 and the power supply 113 can be integrated into a single device. The controller 114 may control the on-off of each of the plasma generating apparatuses 100a-100f, the frequency of the power supply 113, etc.

In some embodiments, as shown in FIG. 4, the plasma treatment system 400 may include gas supply device 410. The gas supply device 410 may include gas source 411, such as a gas storage tank and a gas transmission tube. The gas source 411 may be connected to gas inlet 109 of each of the plasma generating apparatuses 100a-100f to communicate with plasma generating chamber 106 of each of the plasma generating apparatuses 100a-100f to supply gas to the plasma generating chamber 106.

In some embodiments, as shown in FIG. 4, the gas supply device 410 may further include filter 412 between the gas source 411 and the gas inlet 109 of each of the plasma generating apparatuses 100a-100f. The filter 412 can filter the gas from the gas source 411 to filter possible dust particles and impurities and improve the purity of the gas entering the plasma generating chamber 106.

In some embodiments, as shown in FIG. 4, the gas supply device 410 may further include needle valve 413 and gas inlet valve 414 between the gas source 411 and the gas inlet 109 of each of the plasma generating apparatuses 100a-100f. The needle valve 413 and the gas inlet valve 414 may be configured to control the flow velocity of the gas from the gas source to maintain the plasma generating chamber 106 or plasma treatment chamber 420 of each of the plasma generating apparatuses 100a-100f at an appropriate pressure.

In some embodiments, the plasma treatment system 400 may include the plasma treatment chamber 420, as shown in FIG. 4. The plasma treatment chamber 420 may include a space for accommodating the article to be treated by the plasma. Each of the plasma generating apparatuses 100a-100f is fixedly connected to the plasma treatment chamber 420, for example, through a flange of base 110, such that the plasma generating chamber 106 communicates with the accommodating space of the plasma treatment chamber 420. During operation, one or more (e.g., all) of the plasma generating apparatuses 100a-100f are started to generate plasma in the plasma generating chamber 106. Charged particles and neutral activated particles (such as neutral excited state atoms, molecules, and free radicals) in the plasma are separated. The neutral activated particles can be diffused to the plasma treatment chamber 420 to perform plasma treatment on the article in the accommodating space.

In some embodiments, as shown in FIG. 4, the plasma treatment chamber 420 may include chamber door 421. The article to be treated by the plasma may enter or exit the plasma treatment chamber 420 through the chamber door 421. In some embodiments, the chamber door 421 may use a fast-door design.

In some embodiments, as shown in FIG. 4, the plasma treatment chamber 420 may include vacuum gauge 422 configured to detect a vacuum degree in the plasma treatment chamber 420.

In some embodiments, the plasma treatment system 400 may include vacuum device 430, as shown in FIG. 4. The vacuum device 430 may include vacuum pump 431 and vacuum valve 432. The vacuum pump 431 is connected to the plasma treatment chamber 420 to communicate with the accommodating space of the plasma treatment chamber 420. The vacuum pump 431 may control the vacuum degree in the accommodating space of the plasma treatment chamber 420. The vacuum valve 432 is provided between the vacuum pump 431 and the plasma treatment chamber 420 and configured to control the on-off of the vacuum pump.

In some embodiments, the plasma treatment system 400 may include vacuum release device 440, as shown in FIG. 4. The vacuum release device 440 may be connected to the plasma treatment chamber 420. The vacuum release device 440 may include gas release valve 442 configured to control the opening and closing of a gas release channel. In some embodiments, the vacuum release device 440 may include filter 441 configured to filter a gas entering the plasma treatment chamber 420 from the vacuum release device 440. The filter 441 can filter possible dust particles and impurities in the gas, which can improve the stability of the plasma treatment system 400 to prolong the service life and prevent secondary pollution to the article in the plasma treatment chamber 420.

In some embodiments, the plasma treatment system 400 may include a system controller (not shown). It should be understood that the system controller may be integrated with the controller 114, or the controller 114 may serve as the system controller. The system controller may be connected to one or more components of the plasma treatment system 400 and configured to control the connected component. For example, the system controller may be connected to each of the plasma generating apparatuses 100a-100f and the gas supply device 410 and configured to control the start and maintenance of the plasma actuator. When the pressure in the plasma generating chamber 106 or the plasma treatment chamber 420 is lower than a preset value, the system controller opens the gas inlet valve 414 or increases the opening of the needle valve 413. When the pressure in the plasma generating chamber 106 or the plasma treatment chamber 420 is higher than a preset value, the system controller closes the gas inlet valve 414 or decreases an opening of the needle valve 413. In this way, the system controller can maintain the pressure in the plasma generating chamber 106 or the plasma treatment chamber 420 at the preset value or within the preset range.

In some embodiments, the system controller of the plasma treatment system 300 or the plasma treatment system 400 may include a universal processor or a specific processor. The system controller may further include a memory configured to store a set of instructions. These instructions may be read and executed by the processor to control the plasma treatment system 300 or 400.

In some embodiments, the article to be treated by the plasma may be put into the plasma treatment chamber 320 or 420 through the chamber door 321 or 421 of the plasma treatment system 300 or 400. The system controller of the plasma treatment system 300 or 400 may start the vacuum device 330 or 430 to vacuumize the plasma treatment chamber 320 or 420. When the pressure in the plasma treatment chamber 320 or 420 reaches or gets close to the preset value, the system controller can start the gas supply device 310 or 410 (for example, by adjusting the needle valve 313 or 413 or the switching valve 314 or 414) to maintain the pressure in the plasma treatment chamber 320 or 420 at the set value or within the preset range.

The system controller may start the plasma generating apparatus 100 or the plasma generating apparatuses 100a-100f. For example, the system controller may start the power supply 113, such that the feed-through conductor 101 feeds power to the cylindrical electrode 102. The cylindrical electrode 102 ionizes the gas in the plasma generating chamber 106 to generate the plasma. Charged particles and neutral activated particles (such as neutral excited state atoms, molecules, and free radicals) in the plasma are separated. The neutral activated particles can be diffused to the plasma treatment chamber 320 or 420 to perform the plasma treatment on the article.

The system controller may shut down the plasma generating apparatus 100 or the plasma generating apparatuses 100a-100f, the gas supply device 310 or 410, and the vacuum device 330 or 430 when a predetermined condition occurs. The predetermined condition includes a case where a predetermined time expires, a case where a parameter (such as a parameter for characterizing physical, biological, and chemical properties of the article) reaches a predetermined value, etc.

The system controller may start the vacuum release device 340 or 440. For example, the system controller can open the gas release valve 342 or 442, such that the pressure in the plasma generating chamber 106 and the plasma treatment chamber 320 or 420 rises and restores to atmospheric pressure. The article treated by the plasma can be taken out.

The system controller can automatically control the plasma treatment system 300 or the plasma treatment system 400, which improves the efficiency of the plasma treatment, increases the throughput of the plasma treatment, and can realize the plasma treatment on a batch of particles.

In some embodiments, the plasma generating apparatus (such as the plasma generating apparatus 100 in FIG. 3 or the plasma generating apparatuses 100a-100f in FIG. 4) may be located outside the plasma treatment chamber (such as the plasma treatment chamber 320 in FIG. 3 or the plasma treatment chamber 420 in FIG. 4). With the remote plasma source, the plasma generated by the plasma generating apparatus is diffused to the plasma treatment chamber to treat the article in the plasma treatment chamber. Therefore, the plasma treatment system can prevent interference of the plasma generating apparatus with the plasma treatment chamber and is applied to the large-volume plasma treatment chamber. For example, since the electrode is not provided in the plasma treatment chamber, the plasma treatment system can overcome the limitations of the conventional ionization method regarding the size and structure of the electrode. In some embodiments, a single plasma generating apparatus can be applied to a plasma treatment chamber having a volume of up to 2 m³ to realize the large-volume plasma treatment.

In some embodiments, in the plasma generated by the plasma generating apparatus (such as the plasma generating apparatus 100 in FIG. 1 or FIG. 3 or the plasma generating apparatuses 100a-100f in FIG. 4), the charged particles have a mean free path of a millimeter level, while the neutral activated particles have a mean free path of up to at least 1.4 m. Hence, the neutral activated particles can be diffused to the plasma treatment chamber without being limited by the structure and size of the article treated by the plasma, thereby achieving the desired treatment effect. Moreover, from common bacteria to hardest-to-kill spores in the field of sterilization and disinfection, the plasma generating apparatus is quite effective, and thus, can realize broad-spectrum sterilization. In some embodiments, in response to the plasma treatment, the number of bacteria or viruses of the article to be treated by the plasma is typically decreased by one order of magnitude every minute and a sterility assurance level (SAL) of 10⁻⁶ can be realized within 10 minutes, all of which contributes to quick sterilization and disinfection.

Since the plasma treatment depends on the physical or chemical properties of active particles, there are neither toxic by-products nor toxic residues, and therefore, the plasma treatment is friendly to environments and operators.

In some embodiments, the plasma generating apparatus (such as the plasma generating apparatus 100 in FIG. 3 or the plasma generating apparatuses 100a-100f in FIG. 4) in the plasma treatment system (such as the plasma treatment system 300 in FIG. 3 or plasma treatment system 400 in FIG. 4) may also use a structure different from that of the plasma generating apparatus 100 in FIG. 1 and may be, for example, the existing plasma generating apparatus and is not limited by the present disclosure.

In some embodiments, the plasma generated by the plasma generating apparatus (such as the plasma generating apparatus 100 in FIG. 1 or FIG. 3 or the plasma generating apparatuses 100a-100f in FIG. 4) is low-temperature plasma. The plasma may be divided into high-temperature plasma and low-temperature plasma. During discharge of the low-temperature plasma, electrons are at a very high temperature, while heavy particles are at a very low temperature, which creates a low-temperature state of the whole system. Therefore, the plasma generating apparatus can be applied to thermo-sensitive materials (such as thermo-sensitive medical appliances). The plasma treatment at a low temperature prevents the high-temperature plasma from destroying the article to be treated by the plasma.

In some embodiments, the plurality of plasma generating apparatuses (such as the plasma generating apparatuses 100a-100f in FIG. 4) in the plasma treatment system (such as the plasma treatment system 400 in FIG. 4) is arranged into an array, such as a one-dimensional (1D) or two-dimensional (2D) array. In this way, the volume of the plasma treatment chamber (such as the plasma treatment chamber 420 in FIG. 4) of the plasma treatment system is not limited. In some embodiments, the plasma treatment chamber of the plasma treatment system can have a volume of at least 10 m³ or even at least 100 m³, which greatly improves the throughput of the plasma treatment system.

It is to be understood that these embodiments described herein are merely exemplary embodiments, rather than limitations to the present disclosure. Any modifications, equivalent substitutions, improvements, and the like made within the spirit and principle of the present disclosure should fall within the protection scope of the present disclosure.

What is claimed is:

1. A plasma generating apparatus comprising:
a feed-through device configured to feed a medium frequency (MF) power; and
a cylindrical electrode comprising a plurality of through holes in a wall of the cylindrical electrode, wherein the cylindrical electrode is connected to the feed-through device and configured to receive the MF power from the feed-through device and ionize a gas to generate plasma.

2. The plasma generating apparatus according to claim 1, wherein the cylindrical electrode comprises:
a main body; and
an end cover fixed to one end of the main body.

3. The plasma generating apparatus according to claim 1, wherein the feed-through device comprises:
a feed-through conductor electrically connected to the cylindrical electrode and configured to feed the MF power to the cylindrical electrode; and
a feed-through insulating layer covering at least a part of the feed-through conductor.

4. The plasma generating apparatus according to claim 1, further comprising:
a housing, wherein the housing defines a plasma generating chamber, and the cylindrical electrode is located in the plasma generating chamber.

5. The plasma generating apparatus according to claim 4, wherein the housing comprises:
a water cooling channel; and
a water inlet and a water outlet both communicating with the water cooling channel.

6. The plasma generating apparatus according to claim 4, wherein the housing comprises:
a gas inlet communicating with the plasma generating chamber and configured to supply the gas to the plasma generating chamber.

7. The plasma generating apparatus according to claim 1, wherein the cylindrical electrode comprises:
a water cooling channel; and
a water inlet and a water outlet both communicating with the water cooling channel.

8. The plasma generating apparatus according to claim 7, further comprising:
a water inlet tube connected to the water inlet of the cylindrical electrode; and
a water outlet tube connected to the water outlet of the cylindrical electrode,
wherein at least a part of each of the water inlet tube and the water outlet tube comprises an insulating tube.

9. The plasma generating apparatus according to claim 1, wherein
the cylindrical electrode comprises one or more of stainless steel, tantalum, aluminum, titanium, molybdenum, niobium, tungsten, or graphite; and/or
the gas comprises one or more of air, oxygen, nitrogen, an inert gas, or liquid vapor; and/or
the MF power has a frequency in a range of 20 kHz-100 kHz; and/or
a cross-section of the cylindrical electrode comprises a circle, a rectangle, a polygon, or an ellipse.

10. A plasma treatment system comprising:
at least one plasma generating apparatus comprising:
a feed-through device configured to feed a medium frequency (MF) power; and
a cylindrical electrode comprising a plurality of through holes in a wall of the cylindrical electrode, wherein the cylindrical electrode is connected to the feed-through device and configured to receive the MF power from the feed-through device and ionize a gas to generate plasma; and
a plasma treatment chamber communicating with the at least one plasma generating apparatus, wherein the plasma is allowed to be diffused to the plasma treatment chamber.

11. The plasma treatment system according to claim 10, wherein the plasma generating apparatus further comprises:
a housing, wherein the housing defines a plasma generating chamber, and the cylindrical electrode is located in the plasma generating chamber.

12. The plasma treatment system according to claim 11, wherein the plasma generating apparatus further comprises:
a base fixedly connected to the housing and configured to fixedly connect the plasma generating apparatus to the plasma treatment chamber.

13. The plasma treatment system according to claim 11, further comprising:
a gas supply device communicating with the plasma generating chamber and configured to supply the gas to the plasma generating chamber.

14. The plasma treatment system according to claim 13, wherein the gas supply device comprises:
a gas source; and
a filter between the gas source and the plasma generating chamber.

15. The plasma treatment system according to claim 10, further comprising:
a vacuum device connected to the plasma treatment chamber and configured to control a vacuum degree of the plasma treatment chamber.

16. The plasma treatment system according to claim 10, further comprising:
a vacuum release device connected to the plasma treatment chamber or the plasma generating apparatus and configured to release a vacuum in the plasma treatment chamber.

17. The plasma treatment system according to claim 16, wherein the vacuum release device comprises:
a filter configured to filter the gas entering the plasma generating apparatus or the plasma treatment chamber from the vacuum release device.

18. The plasma treatment system according to claim 10, further comprising:
a power supply connected to the feed-through device of the at least one plasma generating apparatus and having a power of at least 100 W or at least 1,000 W.

19. The plasma treatment system according to claim 13, further comprising:
a controller, wherein the controller is connected to the plasma generating apparatus, and configured to start the plasma generating apparatus and shut down the plasma generating apparatus in a predetermined condition, the predetermined condition comprising a predetermined time expiring or a parameter reaching a predetermined value; or
the controller is connected to the gas supply device, and the controller is configured to control the gas supply device to maintain a pressure in the plasma generating chamber or the plasma treatment chamber at a preset value or within a preset range.

20. The plasma treatment system according to claim 10, wherein the plasma treatment chamber has a volume of at least 2 m³, at least 10 m³, or at least 100 m³.
